(19) United States
(12) Patent Application Publication
(10) Pub. No.: US 2002/0156410 A1
(43) Pub. Date: Oct. 24, 2002

(54) Scar tissue treatment bandage and method

(76) Inventor: Roger Lawry, Ojai, CA (US)

Correspondence Address: ROBERT J. SCHAAP, 21241 Ventura Boulevard, Suite 188, Woodland Hills, CA, 91364, US

(21) Appl. No.: 09/839,938

(22) Filed: Apr. 19, 2001

(51) Int. Cl. A61F 13/00

(52) U.S. Cl. 602/48; 602/41

(57) ABSTRACT

A burn treatment bandage in which a silicone composition, primarily a silicone gel, is located on a bandage of the type having an adhesive strip capable of being attached to a user's skin along with a gauze portion thereon. The silicone and, particularly, the silicone gel is pre-incorporated into the gauze, such that when one attempts to treat a burn or other scar tissue, the user may merely apply the bandage such that the silicone material is in contact with the scar tissue.

### BACKGROUND OF THE INVENTION

1. Field of the Invention

This invention relates in general to certain new and useful improvements in devices and methods for treating scar tissue resulting from burns and lacerations and, more particularly, to a device in the nature of a bandage which is pre-impregnated with a silicone containing gel for application to scar tissue.

2. Brief Description of Related Art

It is well known that silicone compounds and, particularly, silicone gels are effective in reducing the oppressive and unsightly appearance of scar tissue resulting from burns and lacerations, and, to some extent, for improving the condition of the scared tissue. Although the pathogeneses for such treatment may not be fully understood, the results have been recognized and substantiated. Moreover, there are a number of prior art references which support the use of silicone containing compounds and, particularly, silicone gels for treating the oppressive and unsightly appearance of scar tissue and particularly scar tissue resulting from burns.

The exact pathological mechanism for treatment of hypertrophic scars is not fully know, although the manifestations thereof are observable and have been well established in the literature. See, for example, “Silicone Gel in Scar Treatment” by Karen J. Quinn, Controlled Therapeutics (Scotland Limited East Kilbride, Scotland). Other literature reports the beneficial effects of silicone on certain types of hypertrophic scars. For example, “Management of Evolving Hypertrophic and Keloid Scars” was reported in Elsevier Science, Inc. 1076-0512/95 by James E. Fulton, M.D., Ph.D.

There are also a large number of prior art references which teach of the use of various types of wound dressings and many of which teach specifically of various types of bandages. These bandages conventionally include an elongate strip with adhesive on one surface thereof and with a center gauze piece. Moreover, the adhesive sections extending beyond the gauze piece are covered with releasable backing strips, such that when these releasable backing strips are removed, the adhesive section is exposed allowing the bandage to be applied to the skin of a user.

Representative of some of the prior art references which disclose conventional bandages are U.S. Pat. No. Des. 410,087, dated May 18, 1999; U.S. Pat. No. 4,832,009, dated May 23, 1989; U.S. Pat. No. 4,834,979, dated May 30, 1989; and U.S. Pat. No. 4,838,353, dated Jun. 13, 1989.

There are also several patent references which disclose the actual use of silicone material for treatment of hypertrophic scars and include, for example, U.S. Pat. No. 5,980,923, dated Nov. 9, 1999; U.S. Pat. No. 4,991,574, dated Feb. 12, 1991; U.S. Pat. No. 5,895,656, dated Apr. 20, 1999; U.S. Pat. No. 5,145,933, dated Sep. 8, 1992; U.S. Pat. No. 5,759,560, dated Jun. 2, 1998, as well as others.

At present, there is no bandage which is pre-treated with a silicone gel, such that the bandage, when unwrapped, can be immediately applied to the skin of a user and with the silicone gel disposed in contact with burned tissue on a user. Typically, burn patients will allow the tissue to heal and typically form scars. These scars are often unsightly and mar the appearance of the individual. Consequently, many burn victims attempt to apply a silicone gel to the burned tissue in order to reduce the unsightly appearance of the scar tissue. However, the burn victim must also attempt to thereafter apply a suitable bandage or other dressing to cover the silicone gel.

Very frequently, the burns in question are relatively small, that is they do not extend over extensive parts of the body. Consequently, it would be easy and convenient to apply a bandage of the type having adhesive sections thereon to allow pre-impregnated gel on the gauze of the bandage to be disposed in contact with the burned tissue of the user. Such a device would eliminate the time consuming and burdensome requirement of applying a dressing to the silicone gel after the same has been applied to the scar tissue.

### OBJECTS OF THE INVENTION

It is, therefore, one of the primary objects of the present invention to provide a bandage which is prepared with a silicone gel capable of being immediately releasably adhered to the skin of the user, such that the gel on the bandage will become disposed in contact with scar tissue of the user.

It is another object of the present invention to provide a bandage of the type stated which has a gauze section and adhesive portions extending beyond the gauze section and where the gauze section is pre-impregnated with a silicone gel to allow the silicone gel to be immediately disposed in contact with scar tissue of a user.

It is further object of the present invention to provide a bandage of the type stated which is simple to use and yet effective as any treatment regiment in which a silicone gel is applied to a wound and thereafter a dressing applied to the silicone gel.

It is also an object of the present invention to provide a bandage of the type stated which is simple to use and can be constructed at a relatively low cost.

It is another salient object of the present invention to provide a method of treating burned tissue by application to the skin of the user surrounding that burned tissue a bandage having a pre-incorporated silicone gel material thereon.

With the above and other objects in view, my invention resides in the novel features of form, construction, arrangement and combination of parts and components presently described and pointed out in the claims.

### BRIEF SUMMARY OF THE INVENTION

The present invention primarily relies upon a dressing in the nature of a bandage which is prepared with a silicone gel material so that the silicone gel resides in contact with burned tissue when the bandage is applied.

More specifically, the bandage of the present invention is formed of an elongate strip, preferably of a fabric material, such as a cloth or a thin light weight plastic sheet material and which has an adhesive coating on two end portions of the strip. Applied to the center portion of the strip between the two adhesive sections is a gauze section. In this respect, the bandage thus far adopts somewhat of a conventional bandage construction.

A silicone gel material is applied to the gauze section, such that when the bandage is applied to the skin of the user, the adhesive end portions will releasably attached to the skin of the user and the burned tissue would be covered by the silicone gel on the gauze section. The adhesive sections, such as the end portions of the elongate strip, would preferably be covered by releasable cover strips, such as, for example, conventional plastic releasable cover strips. In this way, a user merely has to remove the cover strips and apply the bandage the area which has the burned tissue, such that the silicone gel is brought into contact with the burned tissue.

The releasable cover strips could also be provided of sufficient length to literally cover the gel when the strips remain in contact with the adhesive sections. In this way, when the cover strips are removed, the adhesive sections as well as the silicone gel is exposed.

This invention possesses many other advantages and has other purposes which may be made more clearly apparent from a consideration of the forms in which it may be embodied. These forms are shown in the drawings forming a part of and accompanying the present specification. They will now be described in detail for purposes of illustrating the general principles of the invention. However, it is to be understood that the following detailed description and the accompanying drawings are not to be taken in a limiting sense.

### BRIEF DESCRIPTION OF THE DRAWINGS

Having thus described the invention in general terms, reference will now be made to the accompanying drawings in which:

FIG. 1 is a perspective view of a bandage constructed in accordance with and embodying the present invention; and

FIG. 2 is a side elevational view of the bandage of FIG. 1 and showing portions of the releasable cover strip partially removed from the bandage.

### DETAILED DESCRIPTION OF A PREFERRED EMBODIMENT

Referring now in more detail and by reference characters to the drawings, which illustrate a preferred embodiment of the present invention, 10 designates a bandage primarily designed for the treatment of burns, such as burned tissue. In this respect, the bandage is highly effective in the treatment of scar tissue resulting from burns and lacerations. The term “burned tissue” is used in a broad sense to encompass both freshly burned tissue as well as scar tissue which has formed as a result of a burn.

As indicated previously, the full pathological functioning of the silicone is not fully understood. However, it is believed that one of the modes of action of the silicone gel on a scar tissue can be due to either physical or chemical parameters. Some of the physical parameters include pressure, temperature, oxygen tension, hydration, or occlusion. Pressure, to some extent, is believed to have some pathological effect in the treatment of the scar tissue. In fact, pressures of 15 to 40 mm/Hg appears to reduce the color and flatten the scar tissue with using treatment within six to twelve months. Thus, and although the bandage of the present invention as herein described does not necessarily provide a consent pressure, it does provide pressure to the scar tissue and this is believe, in part, to have some beneficial effect.

Although temperature may have an effect in the treatment of scar tissue, again the bandage of the invention does not provide full temperature control. However, it does serve to keep the scar tissue close to body temperature, since the scar tissue is not otherwise exposed to the ambient temperature. The silicone gel should desirably have a similar extensibility to human skin tissue, such as, for example, 41.8%. In this way, the silicone gel can be used over joints without interfering with movement.

The silicone gel has been found to be improbable to organisms tested including standard organisms, such as candida spp. the silicone gel itself actually works as a highly effective bacterial barrier and has the desired mechanical characteristics, such that it is impermeable to liquid water and allow gas exchange. Moreover, it is relatively comfortable for use on patients.

The bandage of the present invention is effective in that it is readily removable pain free or with a minimum discomfort. Moreover, the silicone precludes the sticking of the bandage to the scar tissue. It has been found that elastomeric sheets material used on the portion of the bandage facing the scar tissue is effective. One type of silicone sheeting which is highly effective is a soft polydimethyl siloxane. Moreover, the gel should have a thickness when applied to the surface of the bandage roughly of about 0.05 inch to about 0.3 inch and, preferably, about 0.12 inch to about 0.20 inch. The gel should also have a low modulus of elasticity and thereby promoting skin contact. Another type of silicone gel which can be used is a relatively stiff silicone elastomer offered by Deganin Silicone Ltd. and which should be preferably applied and of a thickness of about 0.01 to about 0.06 inches. In either case, the silicone gel should be relatively occlusive and impermeable to water, since a water accumulating silicone would interfere with patient comfort. Maximum advantage from the silicone elastomer is obtained if substantially coextensive with a gel sheet. In this way, the gel sheet can be applied to the lower surface of the gauze forming part of the bandage. The silicone elastomer film can be fabricated from any silicone elastomer forming composition as, for example, the cross-linking of a polydiorganosiloxane with exposure to electromagnetic radiation or by addition of a carrier agent, such as an organic peroxide. The addition of a cross-linking agent, such as an alkoxy silane or alkylhydrogen polysiloxane and catalyst enabling the cross-linking reaction.

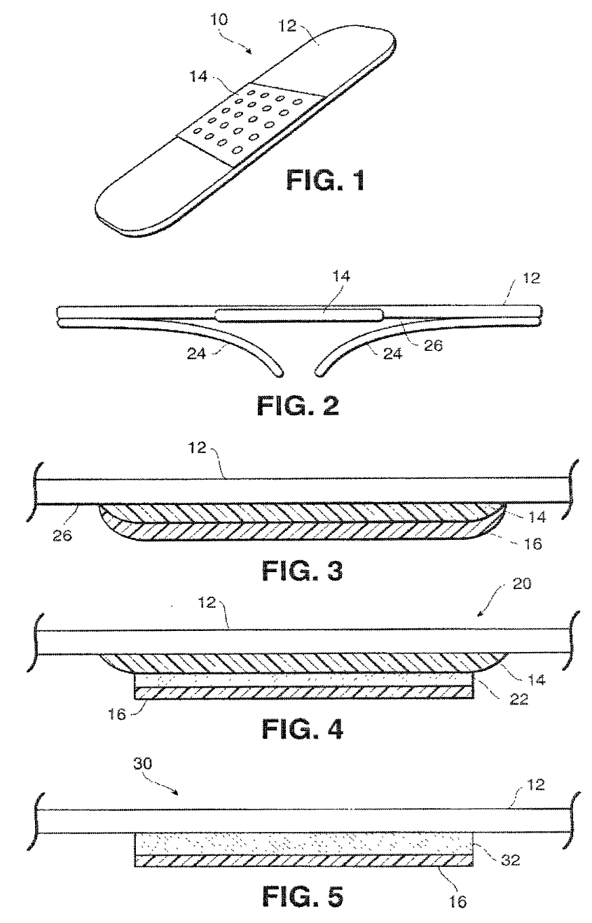

Referring to FIG. 1, a bandage 10 in accordance with the present invention is illustrated and comprises a suitable backing sheet, such as a plastic or plasticized paper sheet 12. Located on the undersurface of the backing sheet 12 is a gauze pad 14. The silicone gel in accordance with the present invention is typically applied to the underside of the gauze 14 as a simple layer 16. In this way, when the bandage is applied to a burn tissue, the silicone gel 16 will come into contact with the scar tissue.

FIG. 4 illustrates an alternate embodiment of a bandage 20 in accordance with the present invention. In this case, like reference numerals which were used to designate components in FIGS. 1-3 will be used to designate similar components in FIG. 4.

The bandage 20 also includes a gauze pad 14 secured to the underside of the backing sheet 12. Moreover, an elastomeric gel sheet 22 may be secured to the underside of the gauge 14. This sheet 22 is relatively thin and almost of unnoticeable thickness in the overall bandage. In this case, the silicone gel layer 16 is applied to the underside of the elastomeric layer 22, as best shown in FIG. 4 of the drawings. In either case, the bandage is highly effective when applied to burn tissue.

The bandage is provided with releasable cover strips 24 which cover an adhesive layer 26 on the underside of the backing sheet 12 beyond the region of the gauge 14. The actual adhesive layer is not specifically illustrated in FIGS. 2-4, although it is commonly present. Moreover, the release strips 24 are readily and easily removable from the adhesive layer on the backing sheet 12.

The release strips 24 are preferably formed of a relatively thin plastic sheet material, such as a vinyl material. In this way, the release strips will not adhere to the silicone gel layer and can be easily and readily removed from the underside of the bandage without disturbing the silicone gel layer. After removal of the cover strips, the bandage can then be applied to the skin of the user with the pad 14 lying over the burn tissue.

It is also possible to use a bandage which is relatively transparent. The particular silicone gel involved may frequently be of a transparent or colorless nature. In this way, the bandage itself and the gel which is used will be relatively unnoticeable, particularly when used in exposed areas, such as the face of an individual or the like.

FIG. 5 illustrates yet another embodiment of a bandage 30 in accordance with the present invention and which includes a backing sheet 12, as shown. Secured to the underside of the backing sheet 12 is a relatively thin foam pad 32. In this case, a light weight open cell foam can be provided for this purpose. In this case, the foam would actually replace the actual gauge pad 14. If desired, a gauge pad 14 could be applied to the undersurface of the foam pad 32. In either case, a lower surface of a silicone gel 16 is then provided for application to the burn tissue of the user.

In one desirable aspect of the invention, it may be preferable to use a silicone adhesive gel in the nature as the adhesive layer. This adhesive differs dramatically from the glues typically used for securement of dressings, such as the polymer glues or hot melt glues commonly used in many adhesive bandages. The adhesive in the present invention as a silicone gel adhesive would be much softer and has a better wetting ability than the conventional glues.

The silicone is located on the dressing portion of the bandage and since a pressure sensitive adhesive is used on the remaining portions of the bandage, in many cases it can actually be reusable. The silicone is highly effective, in that it will operate effectively at another location. Moreover, the silicone itself does not promote bacterial growth.

It is possible to use differing sizes of bandages for the particular scars, such as large bandages for large sized scars, etc.

One of the important aspects of the present invention is that it is frequently difficult to apply silicone to a sharply contoured portion of a person's body as, for example, at the chin or the like. The bandage of the present invention facilitates the actual application of the silicone, since it holds silicone itself and allows the bandage to applied to this relatively small radius or short contour. Thus, the bandage of the present invention is highly effective, reusable, and capable of being produced at a relatively low unit cost.

Thus, there has been illustrated and described a unique and novel burn treatment bandage and method and which thereby fulfills all of the objects and advantages which have been sought. It should be understood that many changes, modifications, variations and other uses and applications which will become apparent to those skilled in the art after considering the specification and the accompanying drawings. Therefore, any and all such changes, modifications, variations and other uses and applications which do not depart from the spirit and scope of the invention are deemed to be covered by the invention.

Having thus described the invention, what I desire to claim and secure by Letters Patent is:

1 A bandage for application to skin surrounding scar tissue on a patient, said bandage comprising:
a) an elongate backing sheet;
b) a relatively soft pliable dressing pad secured to the undersurface of the backing sheet;
c) a silicone gel applied to said dressing pad and capable of being disposed directly in contact with scar tissue when the bandage is applied to adjacent skin tissue of the user; and
d) an adhesive layer on an undersurface of the said backing strip enabling said bandage to be releasably secured to the skin of the user.

2 The bandage of claim 1 further characterized in that said dressing pad is a piece of gauge.

3 The bandage of claim 1 further characterized in that the dressing pad is a foam pad.

4 The bandage of claim 1 further characterized in that the adhesive on the undersurface of the backing strip is covered by releasable strips.

5 The bandage of claim 1 further characterized in that the silicone adhesive layer is secured to a gel sheet before application to the underside of the silicone dressing pad.

6 The bandage of claim 1 further characterized in that said bandage has a length of substantially equivalent to a conventional bandage.

7 A method of treating scar tissue on the body of a patient with a silicone gel, said method comprising:
a) applying a silicone gel to a bandage having an adhesive section thereon;
b) initially covering said silicone gel layer and said adhesive layer;
c) removing cover strips which initially cover the adhesive layer and silicone gel layer; and
d) applying the bandage to the skin of a user with the silicone gel layer overlaying scar tissue and with the adhesive layer being secured to adjacent tissue.
